SEQUENCE LISTING (8 sequences)

SEQ ID NO: 1 - DNA, 23 bp, Artificial Sequence
  PCR primer
  primer_bind (1)..(23): NeuroD, forward primer
ctgatctggtctccttcgtacag

SEQ ID NO: 2 - DNA, 21 bp, Artificial Sequence
  PCR primer
  primer_bind (1)..(21): NeuroD, reverse primer
gatgcgaatggctatcgaaag

SEQ ID NO: 3 - DNA, 20 bp, Artificial Sequence
  PCR primer
  primer_bind (1)..(20): Sox1, forward primer
aaagtcaaaacgaggcgaga

SEQ ID NO: 4 - DNA, 20 bp, Artificial Sequence
  PCR primer
  primer_bind (1)..(20): Sox1, reverse primer
aagtgcttggacctgcctta

SEQ ID NO: 5 - DNA, 20 bp, Artificial Sequence
  PCR primer
  primer_bind (1)..(20): nestin, forward primer
aacagcgacggaggtctcta

SEQ ID NO: 6 - DNA, 20 bp, Artificial Sequence
  PCR primer
  primer_bind (1)..(20): nestin, reverse primer
ttctcttgtcccgcagactt

SEQ ID NO: 7 - DNA, 25 bp, Artificial Sequence
  PCR primer
  primer_bind (1)..(25): beta-actin, forward primer
tggcaccacaccttctacaatgagc

SEQ ID NO: 8 - DNA, 25 bp, Artificial Sequence
  PCR primer
  primer_bind (1)..(25): beta-actin,reverse primer
gcacagcttctccttaatgtcacgc

(19) United States
(12) Patent Application Publication
(10) Pub. No.: US 2017/0247660 A1
(43) Pub. Date: Aug. 31, 2017

(54) PROGENITOR CELLS, METHOD FOR PREPARATION THEREOF AND USES THEREOF

(71) Applicants: Lu-Hai WANG, Miaoli County (TW); National Health Research Institutes, Zhunan Township (TW)

(72) Inventors: Lin-Ju YEN, Zhunan Town (TW); Ko-Jiunn LIU, Zhunan Town (TW); Men-Luh YEN, Taipei (TW)

(73) Assignee: National Health Research Institutes, Zhunan Township (TW)

(21) Appl. No.: 15/512,489

(22) PCT Filed: Sep. 16, 2015
(86) PCT No.: PCT/US2015/050418

§ 371 Date: Mar. 17, 2017

Related U.S. Application Data
(60) Provisional application No. 62/052,088, filed on Sep. 18, 2014.

(51) Int. Cl. C12N 5/071; A61K 35/48; C12N 5/0775

(52) U.S. Cl. CPC C12N 5/0682; C12N 5/0668; A61K 35/48; C12N 2509/00

(57) ABSTRACT

The present application provides progenitor cells and a preparing method thereof. The preparing method comprises obtaining a tissue sample containing myometrium from uterus, treating the tissue sample with collagenase, and culturing the treated tissue sample to obtain the progenitor cells, wherein the progenitor cell is multipotent and immunomodulatory. The present application also provides a method for treating a degenerative disease, an ischemic disease or a disease caused by abnormal immune response comprising administering the progenitor cells to a patient subjecting the disease.

### CROSS-REFERENCE TO RELATED APPLICATION(S)

This application claims priority of U.S. provisional application No. filed 62/052,088 on Sep. 18, 2014.

### BACKGROUND OF THE INVENTION

1. Field of the Invention

The present invention relates to multipotent progenitor cells, a method for preparing the progenitor cells from uterine corpus, and uses of the progenitor cells.

2. Description of the Related Art

Progenitor cell therapy is likely the best answer to curing degenerative diseases such as Parkinson's disease and ischemic diseases such as stroke and myocardial infarction—all disease entities highly correlated with aging populations. Current sources of human progenitor cells (also known as stem cells) include pluripotent stem cells (PSCs) such as human embryonic stem cells (hESCs) and induced pluripotent stem cells (iPS), as well as adult stem cells (ASCs) such as bone marrow mesenchymal stem cells (BMMSCs) and neural stem cells. However, each source is not without its drawbacks for clinical use: PSCs have ethical and tumorigenicity concerns (Thomson, J. A., Itskovitz-Eldor, J., Shapiro, S. S., Waknitz, M. A., Swiergiel, J. J., Marshall, V. S., and Jones, J. M. (1998). Embryonic stem cell lines derived from human blastocysts. Science 282, 1145-1147; Yamanaka, S. (2009). A fresh look at iPS cells. Cell 137, 13-17), while ASCs are very rare cells requiring invasive procedures for procurement with cell numbers and stem cell functions decreasing with age (Rao, M. S., and Mattson, M. P. (2001). Stem cells and aging: expanding the possibilities. Mech Ageing Dev 122, 713-734) and ex vivo expansion (Ho, P. J., Yen, M. L., Tang, B. C., Chen, C. T., and Yen, B. L. (2013). H2O2 accumulation mediates differentiation capacity alteration, but not proliferative decline, in senescent human fetal mesenchymal stem cells. Antioxid Redox Signal 18, 1895-1905) (“Ho et al”)).

Uterus is an organ in the female reproductive tract whose function is to carry the human fetus and nurture until term. Capable of intense growth to accommodate the physiological demands of pregnancy, the uterus corpus is mainly comprised of smooth muscle cells—the myometrium—and is highly vascularized. Removal of the uterus, or a hysterectomy, is the most common surgical procedure performed on non-pregnant woman; in the United States, one of three women past the age of 60 has had a hysterectomy (Schaffer, J. I., and Word, A. (2002). Hysterectomy—still a useful operation. N Engl J Med 347, 1360-1362; Carlson, K. J., Nichols, D. H., and Schiff, I. (1993). Indications for hysterectomy. N Engl J Med 328, 856-860; Frequently Asked Questions: Hysterectomy. (2009). Office on Women's Health, U.S. Department of Health & Human Services).

Ono et al. reported cells termed “myometrial side-population stem cells (myoSPs)” can be isolated from human uterus (Ono, M., Maruyama, T., Masuda, H., Kajitani, T., Nagashima, T., Arase, T., Ito, M., Ohta, K., Uchida, H., Asada, H., et al. (2007). Side population in human uterine myometrium displays phenotypic and functional characteristics of myometrial stem cells. Proc Natl Acad Sci USA 104, 18700-18705 (“Ono et al, PNAS 2007”)). Myometrial tissue has to be incubated in medium with minimal enzyme addition (0.02%) for 4-16 hours with subsequent filtration (2 times) and gradient selection (Ficollpaque), then further trypsinized. MyoSPs were characterized by the ability to efflux Hoechst 33342 dye, which show up on flow cytometric analysis as a “side population,” and also exibit CD31 (+), CD34(+), and CD44(−). However, MyoSPs are unable to differentiate into chondrogenic cells, nor reported to differentiate into neurogenic cells.

Galvez et al., In Vivo 2009, WO2010/057965 and WO2011/042547A1 disclose myometrial-derived mesenchymal stem cells and isolation method thereof. The isolated cells termed “adult myometrial precursors (AMPs)” which were isolated to from myometrial tissue. The myometrial tissue was incubated in serum-free medium and only fragments with small vessels were selected for further culturing. Therefore, the method is for selecting endothelial-like cells, and the results showing high CD31(+) percentage of these isolated AMPs (>99.7% for mouse AMPs and >90% for human AMPs) can be explained accordingly. The cell surface marker profile of AMPs are CD73(−), CD31(+) and HLA-DR (+). Only differentiation capacity of mouse AMPs were disclosed and the mouse AMPs possess osteogenesis, adipogenesis and neurogenesis, but no chondrogenesis was reported.

Therefore, it still needs an alternative source for obtaining progenitor cells and a method for preparing the progenitor cells.

### SUMMARY

The present application describes a method for preparing progenitor cells comprising obtaining a tissue sample containing myometrium from uterus, treating the tissue sample with an enzyme to remove fibrous tissue, and culturing the treated tissue sample to obtain the progenitor cells, wherein the progenitor cell is multipotent and immunomodulatory.

The present application also provides a progenitor cells obtained from the above method.

The present application further provides a method for treating a degenerative disease, an ischemic disease or a disease caused by abnormal immune response comprising administering progenitor cells prepared according to the above method to a patient subjecting the disease.

### BRIEF DESCRIPTION OF THE DRAWINGS

FIG. 1 shows characterization of myometrium-derived multipotent progenitors (MDMPs). (A) Proliferative potential of MDMPs compared to adipose tissue-derived stem cells. (B) MDMPs are negative for side-population (SP) cells. To assay for SP cells, MDMPs were incubated with the Hoechst 33342 dye in the presence or absence of verapamil which blocks efflux of the Hoechst 33342 dye, an activity of SP cells. Propidium iodide at a final concentration of 2 Ag/mL was added to the cells to gate for viable cells, and flow cytometric analysis was performed, with the Hoechst 33342 dye excited at 357 nm and its fluorescence was dual-wavelength analyzed (blue, 402-446 nm; red, 650-670 nm). (C) Surface marker profiling of MDMPs by flow cytometry (gray-filled curve, isotype control; black unfilled line, denoted antibody. (C) Immunofluorescent staining of various markers in MDMPs; scale bar 100m.

FIG. 2 shows the multilineage differentiation capacity of MDMPs. (A) Differentiation capacity of MDMPs into osteogenic (Alizarin Red staining), chondrogenic (Alcian Blue staining), adipogenic (Oil Red O staining), and neurogenic lineages (phase-contrast). Scale bar, 200 μm. (B) Further characterization of neurogenic differentiation potential of MDMPs. Gene expression of neural lineage genes (as denoted in graph) was analyzed by real-time PCR after MDMPs were cultured in 2 neurogenic induction conditions: serum-free medium with retinoic acid (RA; 0.1 μM) or complete medium with Y27632 (Y; 10 μM), an inhibitor of RhoA kinase. Ctl, control (complete medium); *, p<0.05, compared to ctl.

FIG. 3 shows in vitro and in vivo immunomodulatory characteristics of MDMPs. (A) MDMPs suppress stimulated human peripheral blood mononuclear cell to (PBMC) proliferation and more profoundly than bone marrow mesenchymal stem cells (BMMSCs). Human PBMCs were first stained with carboxyfluorescein succinimidyl ester (CFSE)—a green fluorescence dye—to track for cell division after stimulation with phytoagglutinin (PHA) or anti-CD3/28 beads (α-CD3/28) which more specifically stimulates T lymphocytes. Activated CFSE-stained PBMCs were then co-cultured without or with BMMSCs or MDMPs and analyzed by flow cytometry to assess for cell division with low-CFSE staining representing low proliferating PBMCs (% denoted in blue). Left-side histograms denote representative data and right-side graph denote pooled results. (B) MDMPs can suppress both CD4 and CD8 T lymphocyte proliferation, and more significantly than BMMSCs. CD4 and CD8 T lymphocytes were first selected using magnetic beads, stained for CFSE, stimulated with a-CD3/28, co-cultured without or with BMMSCs or MDMPs, and analyzed by flow cytometry for low-CFSE staining T cells (% denoted in blue). Left-side histograms denote representative data and right-side graph denote pooled results with n=4 for each group. (C) Experimental strategy for establishing in vivo inflammatory conditions in wildtype C57BL/6J mice with adoptive transfer of human BMMSCs or MDMPs. Lipopolysaccharide (LPS) challenge was given intraperitoneally (i.p.) with cells transferred thereafter. On Day 3 after LPS challenge, mice were sacrificed and lymphocytes isolated from spleen and regional lymph nodes for assessment of T cell subpopulations of type 1 CD4 cells (Th1) and regulatory T cells. (D) MDMPs suppress interferon-γ (IFN-γ)-expressing Th1 cells and (E) induce CD25high/Foxp3+ regulatory T cells more significantly BMMSCs in vivo. *, p<0.05; **, p<0.01.

### DETAILED DESCRIPTION OF THE EMBODIMENTS

The present application describes a method for preparing progenitor cells comprising obtaining a tissue sample containing myometrium from uterus. The isolated progenitor cell is multipotent and immunomodulatory.

As used herein, the term “myometrium” refers to tissue derived from the middle layer of the uterine wall.

As used herein, the term “uterus” encompasses the cervical canal and uterine cavity.

The multipotent and immunomodulatory progenitor cells of the present application can be obtained from any tissue sample containing myometrium of any suitable source from any suitable animal. In one embodiment, the suitable animal is an mammal such as a rodent, primate, carnivora, artiodactyla and the like, preferably a primate.

In one embodiment, the tissue sample can be obtained from non-pathological post-natal uterus.

In one preferred embodiment, the tissue sample is obtained from the human uterine corpus.

In one embodiment, the uterus can be from hysterectomies. Hysterectomy is the most common surgical procedure performed on non-pregnant woman. The method of the present application allows for the efficient isolation of uterine myometrium-derived multipotent progenitors (MDMPs) from post-hysterectomy specimens—which is to be discarded—with multilineage differentiation capacity, immunomodulation, and high proliferative potential. In addition, the method of the present application is to prepare the progenitor cells from commonly available surgical to ‘waste’ has high therapeutic applicability, since no further invasive procedure need to be performed and no ethical concerns are raised.

In the method of present application, the tissue sample containing myometrium is treated with an enzyme to remove fibrous tissue. It is one-step enzyme treatment, and none of further steps such as filtration and gradient selection is needed. In one embodiment, the enzyme includes collagenase. The enzyme-treated tissue sample is then cultured in a serum-supplemented medium to obtain the progenitor cells. In one embodiment, the treated tissue sample is cultured in a complete medium supplemented by a serum and an antibiotic.

The present application also provides progenitor cells obtained from the above method. The progenitor cells are unique, differing from prior arts in terms of isolation method, differentiation capacity and cell surface marker expression profile.

The progenitor cells have negative expression of cell surface marker CD34, i.e. CD34(−). In one embodiment, the progenitor cells have positive expression of cell surface marker CD44, CD73, CD90, CD105, or any combination thereof. In one embodiment, the progenitor cells have negative expression of cell surface marker CD31, CD14, CD45, CD19, HLA-DR, Side Population (SidePop) or any combination thereof. In one preferred embodiment, the progenitor cells have positive expression of cell surface markers CD44, CD73, CD90 and CD105, and negative expression of cell surface markers CD31, CD34, CD14, CD45, CD19, HLA-DR and SidePop.

The progenitor cells of the present application can undergo osteogenesis, adipogenesis, chondrogenesis, and neurogenesis.

The progenitor cells of the present application possess strong immunomodulatory properties, which have suppressive effects on both CD4 and CD8 T lymphocytes.

The progenitor cells of the present application represent a new source of human stem cells which can be isolated without ethical concerns and in high volumes for wide clinical applicability. The clinical applications of the progenitor cells include, but is not limited to, degenerative diseases, ischemic diseases, a disease caused by abnormal immune response and the like.

Therefore, the present application further provides a method for treating a degenerative disease, an ischemic disease or a disease caused by abnormal immune response comprising administering the progenitor cells to a patient subjecting the disease.

In one embodiment, the degenerative disease includes, but is not limited to, Parkinson's disease, Alzheimer's disease, Huntington's disease, cerebral atrophy, cerebellar atrophy, schizophrenia and dementia.

In one embodiment, the ischemic disease includes, but is not limited to, stroke, cerebral apoplexy, cerebral hemorrhage, cerebral infarction, head trauma, vascular dementia and myocardial infarction.

In one embodiment, the disease caused by abnormal immune response includes, but is not limited to, autoimmune disease or graft rejection of organ transplantation. The autoimmune disease includes such as system lupus erythematosus, multiple sclerosis, rheumatoid arthritis, type 1 diabetes mellitus, coeliac disease, Sjögren's syndrome, Hashimoto's thyroiditis, Graves' disease, and idiopathic thrombocytopenic purpura.

In the method, the patient can be an mammal such as a rodent, primate, carnivora, artiodactyla and the like, preferably a primate. In one embodiment, the patient is a human.

Examples for preparing the progenitor cells are described hereinafter.

### EXAMPLES

Methods & Materials

Cell Isolation & Culture

Uteri from hysterectomies for benign disease were obtained with informed consent approved by the institutional review board. The myometrium was dissected and separated from the endometrium and serosa, then digested with collagenase IV (Sigma-Aldrich) for 30 minutes. The samples and supernatant were then cultured in complete medium consisting of Dulbecco's modified Eagle's medium low glucose (Gibco-Invitrogen, Grand Island, USA) supplemented by 10% fetal bovine serum (FBS; selected lots, HyClone, Logan, Utah, USA), 100 U/ml penicillin, and 100 g/ml streptomycin (Sigma-Aldrich, St. Louis, Mo., USA). Cell cultures were maintained at 37° C. with a water-saturated atmosphere and 5% CO2. Medium was replaced one to two times every week, and when 80% confluent, cells were subcultured at 1:3 ratio.

Immunophenotyping

To detect surface antigens, aliquots of cells were washed with PBS containing 2% FBS after detachment with 0.25% trypsin/EDTA. All antibodies were purchased from BD Biosciences (Franklin Lakes, N.J.). Cells were stained with fluorescein isothiocyanate (FITC)- or phycoetrythrin (PE)-conjugated antibodies and compared with appropriate isotype controls. Flow cytometry analysis was performed using a FACSCalibur flow using CellQuest software (BD Biosciences) as we have previously reported (Yen, B. L., Huang, H. I., Chien, C. C., Jui, H. Y., Ko, B. S., Yao, M., Shun, C. T., Yen, M. L., Lee, M. C., and Chen, Y. C. (2005). Isolation of multipotent cells from human term placenta. Stem Cells 23, 3-9; Yen, B. L., Yen, M. L., Hsu, P. J., Liu, K. J., Wang, C. J., Bai, C. H., and Sytwu, H. K. (2013). Multipotent human mesenchymal stromal cells mediate expansion of myeloid-derived suppressor cells via hepatocyte growth factor/c-Met and STATS. Stem Cell Reports 1, 139-151). Sidepopulation cells were determined as previously reported (Ono et al. PNAS 2007) with the use of 50 μm verapamil to block efflux of the Hoechst 33342 dye (Sigma-Aldrich) (Goodell, M. A., Brose, K., Paradis, G., Conner, A. S., and Mulligan, R. C. (1996). Isolation and functional properties of murine hematopoietic stem cells that are replicating in vivo. J Exp Med 183, 1797-1806).

Differentiation Studies and Characterization

Differentiation into adipogenic, osteoblastic, and chondrogenic lineages was performed and characterized as we and others have previously reported (Liu, K. J., Wang, C. J., Chang, C. J., Hu, H. I., Hsu, P. J., Wu, Y. C., Bai, C. H., Sytwu, H. K., and Yen, B. L. (2011). Surface expression of HLA-G is involved in mediating immunomodulatory effects of placenta-derived multipotent cells (PDMCs) towards natural killer lymphocytes. Cell Transplant 20, 1721-1730; Pittenger, M. F., Mackay, A. M., Beck, S. C., Jaiswal, R. K., Douglas, R., Mosca, J. D., Moorman, M. A., Simonetti, D. W., Craig, S., and Marshak, D. R. (1999). Multilineage potential of adult human mesenchymal stem cells. Science 284, 143-147). Neurogenic differentiation was induced by standard methods; briefly, by culturing cells at low density (1000 cells/cm3), in serum-free medium with the addition of 0.5 μm retinoic acid (Sanchez-Ramos, J. R., Song, S., Kamath, S. G., Zigova, T., Willing, A., Cardozo-Pelaez, F., Stedeford, T., Chopp, M., and Sanberg, P. R. (2001). Expression of neural markers in human umbilical cord blood. Exp Neurol 171, 109-115), or in complete medium with the addition of 10 μm Y-27632 as we previously reported (Wang, C. H., Wu, C. C., Hsu, S. H., Liou, J. Y., Li, Y. W., Wu, K. K., Lai, Y. K., and Yen, B. L. (2013). The role of RhoA kinase inhibition in human placenta-derived multipotent cells on neural phenotype and cell survival. Biomaterials 34, 3223-3230 (“Wang et al.”). All reagents are from Sigma-Aldrich with the exception of TGF-β3 for chondrogenic differentiation, which was obtained from R&D Systems (Minneapolis, Minn.).

Immunofluorescence Staining

Immunofluorescence staining for neural characterization was performed as previously reported (Wang et al.). Briefly, cultured cells were fixed with 4% paraformaldehyde (PFA) (Sigma-Aldrich) for 10 minutes at room temperature and permeabilized with 0.1% Triton-X 100 (Sigma-Aldrich) for 10 minutes. Primary antibodies against the human antigens nestin and glial fibrillary acidic protein were purchased from Chemicon (Temecula, Calif.); for α-SMA were purchased from Sigma-Aldrich. Samples were first incubated with the primary antibodies at 4° C. overnight, then rinsed three times with PBS and incubated for 60 minutes at room temperature with FITC-conjugated secondary antibodies at a dilution of 1:100. All samples were stained with 4′,6-Diamidino-2-phenylindole (DAPI, 1:2000; Molecular Probes). Staining was visualized under a fluorescence microscope (Olympus, Tokyo, Japan).

Cell Proliferation Assessment

Cells were seeded initially at 1.5×104 cells/cm2 beginning from the 2nd passage (P2). Upon sub-confluent growth at a density of 80%, cells were trypsinized as usual and replated at the initial density. Growth curve were determined as we previously described (Ho et al.).

Reverse Transcription PCR (RTPCR)

RNA was isolated and RTPCR was performed as we previously reported (Ho et al.). Primers are as follows:
NeuroD, forward primer CTGATCTGGTCTCCTTCGTACAG, reverse primer GATGCGAATGGCTATCGAAAG;
Sox1: forward primer AAAGTCAAAACGAGGCGAGA, reverse primer AAGTGCTTGGACCTGCCTTA, and
nestin: forward primer AACAGCGACGGAGGTCTCTA, reverse primer TTCTCTTGTCCCGCAGACTT.
   Quantification was performed after normalization against the gene product of β-actin (primers: forward TGGCACCACACCTTCTACAATGAGC, reverse GCACAGCTTCTCCTTAATGTCACGC).

Progenitor/Stem Cell and Human Peripheral Blood Mononuclear Cells (PBMCs)/Lymphocyte Coculture Experiments

PBMC-related experiments were carried out similar to our previously described methods (Chang, C. J., Yen, M. L., Chen, Y. C., Chien, C. C., Huang, H. I., Bai, C. H., and Yen, B. L. (2006). Placenta-derived multipotent cells exhibit immunosuppressive properties that are enhanced in the presence of interferon-gamma Stem Cells 24, 2466-2477 (“Chang et al.”); Chen, P. M., Liu, K. J., Hsu, P. J., Wei, C. F., Bai, C. H., Ho, L. J., Sytwu, H. K., and Yen, B. L. (2014) Induction of immunomodulatory monocytes by human mesenchymal stem cell (MSC)-derived hepatocyte growth factor (HGF) through ERK1/2. J Leukoc Biol 95, 295-303 (“Chen et al.”)). Briefly, human PBMCs were isolated from the buffy coat of healthy donor blood samples (Taiwan Blood Services Foundation, Taipei Blood Center, Taipei, Taiwan) obtained with informed consent approved according to the procedures of the institutional review board and cultured as previously reported. Isolated PBMCs were first stained with carboxyfluorescein succinimidyl ester (CFSE; Gibco-Invitrogen)—a green fluorescence dye—to track for cell division after stimulation with phytoagglutinin (PHA; Sigma-Aldrich) or anti-CD3/28 beads (a-CD3/28; Dynabeads) which more specifically stimulates T lymphocytes. Progenitor/stem cells (MDMPs or BMMSCs) were plated at 3.5×104 cells per well in 6-well plates, and incubated at 37° C. for 24 hours prior to co-culture with stimulated PBMCs (1×105). After 3 days further of culture, cells were harvested and evaluated by flow cytometric analysis for CFSE staining intensity or various protein (surface marker, cytokine, transcription factor, etc.) expression.

Mouse In Vivo Experiments

All animal work was performed in accordance with protocols approved by the institutional Animal Care and Use Committee. Wild-type C57BL/6J mice were purchased from the National Laboratory Animal Center of Taiwan (Taipei, Taiwan). Induction of proinflammatory leukocytes in vivo was performed similarly as previously reported (Shi, G., Vistica, B. P., Nugent, L. F., Tan, C., Wawrousek, E. F., Klinman, D. M., and Gery, I. (2013). Differential involvement of Th1 and Th17 in pathogenic autoimmune processes triggered by different TLR ligands. J Immunol 191, 415-423). Briefly, lipopolysaccharide (LPS; 100 μg, Escherichia coli 00041:B4; Sigma-Aldrich) was injected intraperitoneally into 8- to 12-week-old mice, followed 2 hours later by transfer of either MDMPs or BMMSCs (1×105 cells/mouse). Mice were sacrificed on day 3 with harvesting of leukocytes from the spleen and regional lymph nodes for assessment of Th1 cells and Tregs as previously reported (Chen et al; Chang et al).

Results

Characterization of Multipotent Progenitors From the Human Uterine Corpus

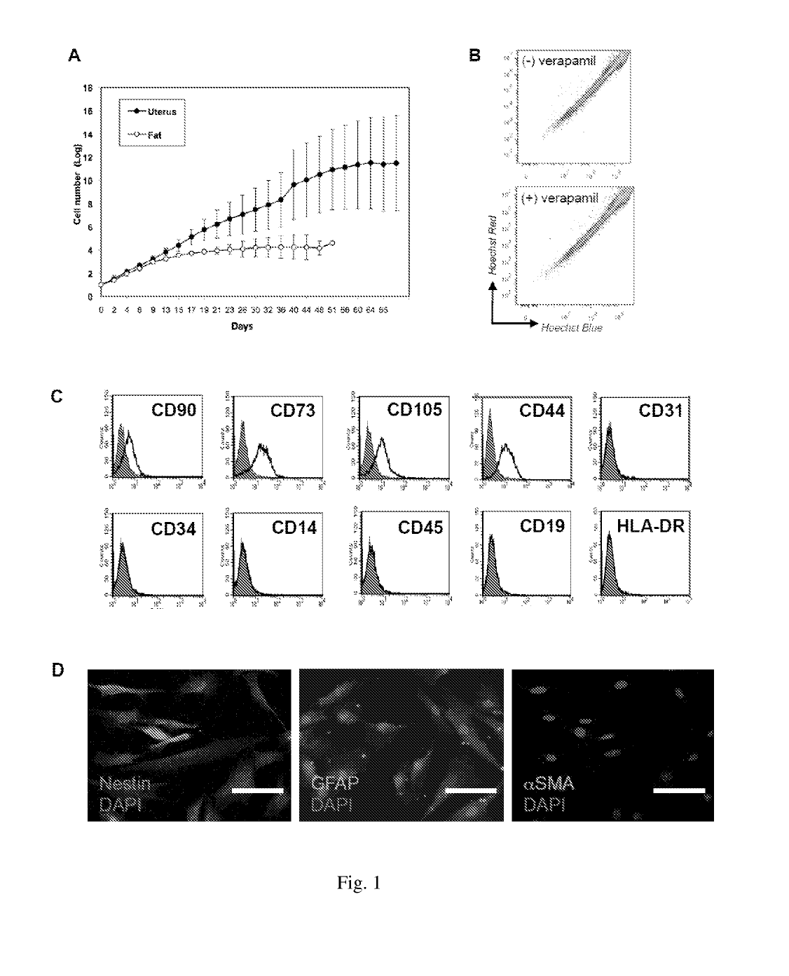

Myometrial-derived multipotent progenitors (MDMPs) were isolated from posthysterectomy specimens of benign diagnosis. These MDMPs are highly roliferative, even as compared to somatic progenitors isolated from adipose tissue, which is currently a popular source for isolation of human therapeutic progenitors (FIG. 1A). Characterization of these MDMPs showed that these progenitors do not efflux Hoechst dye and so do not fit the profile of Side-Population cells (FIG. 1B). In terms of surface marker expression, MDMPs are positive for CD90, CD73, CD105, and CD44, but are negative for the endothelial marker CD31 and a number of hematopoietic markers including CD34, CD14, CD45, CD19, and HLA-DR (FIG. 1C). Interestingly, MDMPs are positive for two neural stem cell markers, nestin and GFAP (FIG. 1D). Moreover, while MDMPs are isolated from an organ comprised of smooth muscle, they are negative for the smooth muscle α-smooth muscle actin (α-SMA) (FIG. 1D), indicating that MDMPs are not comprised of end-differentiated uterine corpus smooth muscle cells.

MDMPs Possess Multilineage Potential

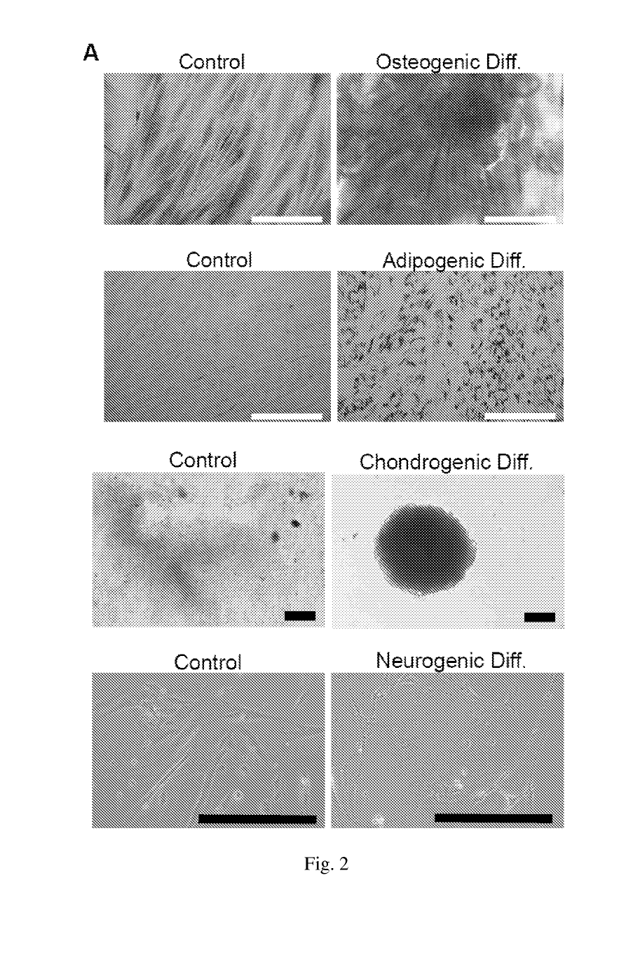

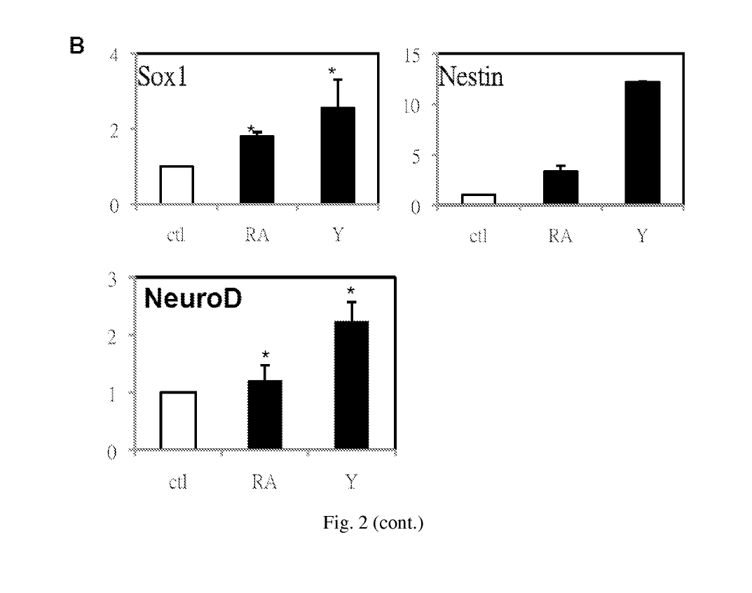

The differentiation potential of MDMPs were then assessed. It was found that MDMPs can differentiate into multiple cell lineages, including osteogenic, hondrogenic, adipogenic, and neurogenic lineages (FIG. 2A). Further characterization of neurogenic differentiation potential of MDMPs show that when cultured in various neurogenic-inducing condition, these progenitors increase expression of a number of neural stem cell-related genes such as Sox1, nestin, and NeuroD (FIG. 2B). Thus, MDMPs possess multilineage differentiation potential and have wide applicability towards osteogenic diseases including fractures, osteoporosis, osteogenesis imperfecta; chondrogenic diseases including osteoarthritis and rheumatoid arthritis; and neurological diseases including stroke, Parkinson's, amyotrophic lateral sclerosis, and dementia.

MDMPs Possess More Significant In Vitro and In Vivo Immunomodulatory Capacity Than BMMSCs

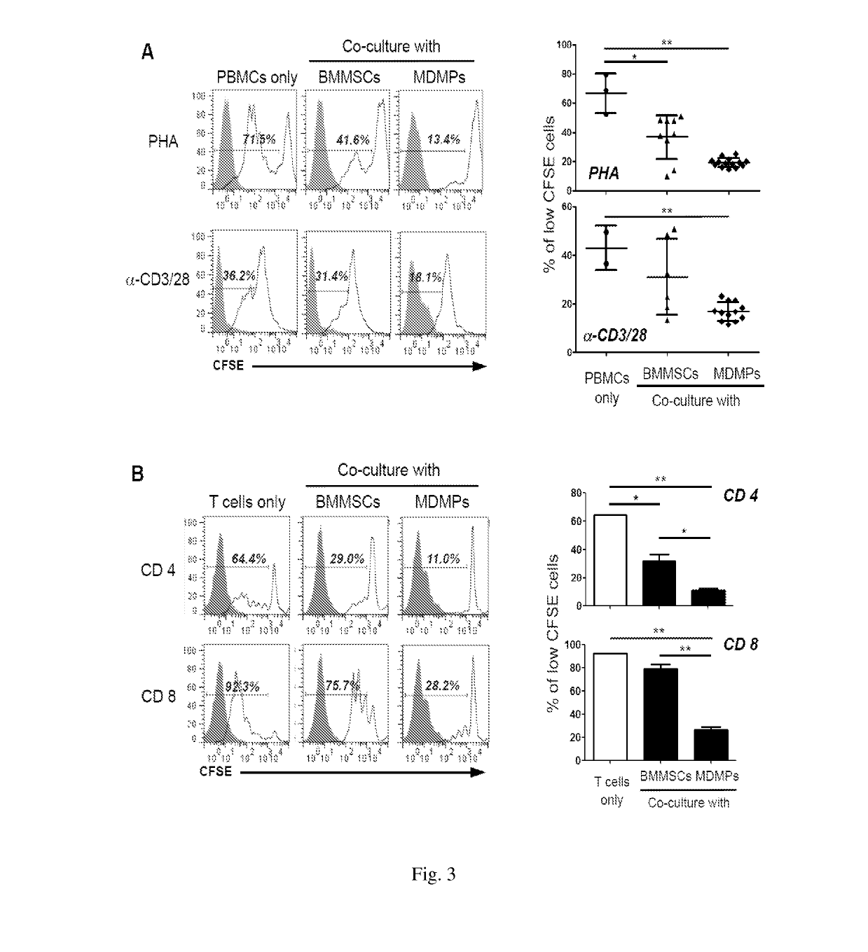

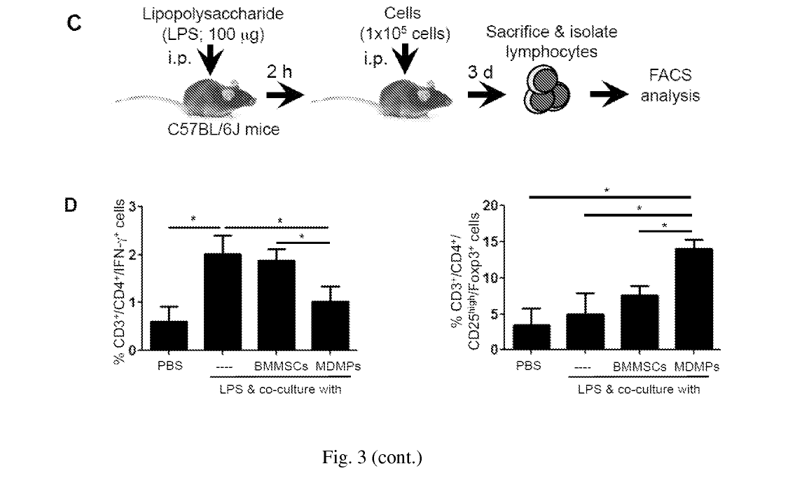

Increasing evidence show that inflammation is involved in multiple disease entities previously not thought to involve such processes, and this includes such epidemiologically prominent diseases including neurodegenerative diseases, ischemic diseases and cancer. The uterus has been postulated to have a unique immunological milieu (Moffett-King, A. (2002). Natural killer cells and pregnancy. Nat Rev Immunol 2, 656-663). Thus, whether MDMPs possess immunomodulatory effects was evaluated. To assess whether MDMPs are immunosuppressive, it was performed mixed lymphocyte reactions using stimulated human peripheral blood monoclear cells (PBMCs) and co-cultured either MDMPs or BMMSCs—a known immunomodulatory type of stem/progenitor cells—simultaneously. It was found that coculture of MDMPs not only suppressed the proliferation of PBMCs stimulated with either phytoagglutinin or anti-CD3/28 beads, but can do so more potently than BMMSCs can be significantly suppressed when MDMPs are co-cultured (FIG. 3A). Moreover, the suppressive effects of MDMPs on both CD4 and CD8 T lymphocytes, two populations of leukocytes critical in mediating a number of immune-related diseases, and these suppressive effects are more significant than BMMSCs. In addition, in an in vivo model of inflammation, more significantly than BMMSCs, MDMPs can significantly inhibit effector T cell function as represented by type 1, interferon-g-secreting CD4 cells (Th1 cells) and enhance immunodulation as represented CD4+/CD25high/Foxp3+ regulatory T cells (FIGS. 3C & D). Thus, MDMPs are highly immunomodulatory and can likely be applied towards therapy of immune- and inflammationrelated diseases, such as autoimmune diseases, inflammatory colitis, organ rejection, neurodegenerative diseases, and ischemic diseases.

Comparison Between the MDMPs and the Prior Arts

Referring to Table 1, summary of the comparison are shown.

Ono et al. (Ono et al, PNAS 2007) disclosed that the MyoSPs were isolated by incubating myometrial tissue in medium with minimal enzyme addition (0.02%) for 4-16 hours with subsequent filtration (2 times) and gradient selection (Ficollpaque), then the tissue was further trypsinized.

Galvez et al., In Vivo 2009, WO2010/057965 and WO2011/042547A1 disclose that AMPs were isolated by incubating the myometrial tissue in serum-free medium, and selecting only fragments with small vessels for further culturing. The method likely selects endothelial-like cells, and the results showing high CD31(+) percentage of these isolated AMPs (>99.7% for mouse AMPs and >90% for human AMPs) can be explained accordingly.

In the method of the present application, a one-step of enzymatic treatment is applied for less than 1 hour without filtration or gradient selection, with culturing in serum-supplemented medium. In addition, there is no need to use portions with vessels for further culturing in the present application.

MyoSPs were characterized by the ability to efflux Hoechst 33342 dye, which show up on flow cytometric analysis as a “side population,” which the present application does not yield (see FIG. 1B). MyoSPs exibit CD31 (+), CD34(+) and CD44(−), AMPs exibit CD31(+), CD73(−) and HLA-DR (+), however, MDMPs of the present application are CD31(−), CD34(−), CD44(+), CD73(+) and HLA-DR(−) (see FIG. 1C).

In addition, MyoSPs are unable to differentiate into chondrogenic cells, nor reported to differentiate into neurogenic cells, as MDMPs can Immunomodulatory capacity of MyoSPs are not discussed by Ono et al., but MyoSPs may unlikely to have this capacity since they are CD34(+) and CD31(+), indicating a hematopoietic background and likely immunogenic.

Only mouse AMPs but not human AMPs were tested for differentiation capacity. The mouse AMPs possessed osteogenesis, adipogenesis and neurogenesis, but no chondrogenesis was reported Immunomodulatory capacity of AMPs are not discussed in the above disclosures, but AMPs could possibly be immunogenic due to being HLA-DR (+) at baseline.

Obviously, MDMPs possesses distinct characteristics from MyoSPs and AMPs.

TABLE 1

| | Isolated cells | | |
|---|---|---|---|
| | | | AMPs |
| | MDMPs | | (Galvez et al, 2009 + |
| Characteristic | (our | MyoSP cells | WO2010/057965 + |
| & Marker profile | method) | (Ono et al, 2007) | WO2011/042547A1) |
| Isolation Method | Enzymatic | Minimal enzyme treatment | Exfolinted fragment |
| | treatment | (0.02%) for 4-16 hours with | “explants” with small |
| | only, <1 hour | subsequent filtration (2 | vessels selected in serum- |
| | | times) and gradient | free medium |
| | | selection (Ficoll-paque) | |
| | | then further trypsinized | |
| CD31* | − | ~67%+ | +>90% both mouse & |
| | | | human AMPs |
| CD34**§ | − | + | +>99% for mouse AMPs, |
| | | | − for human AMPs |
| CD73 | + | + | − |
| CD44 | + | − | + |
| CD105 | + | + | +>50% for human AMPs |
| Side population (Efflux | − | + | Not done |
| of Hoechst 33342 stain) | | | |
| HLA-DR§ | − | Not reported | +~30% for mouse AMPs |
| Osteogenesis | + | + | + for mouse AMPs only |
| Adipogenesis | + | + | + for mouse AMPs only |
| Chondrogenesis | + | Not possible (stated in | Not reported |
| | | publication) | |
| Neurogenesis | + | Not reported | + for mouse AMPs only |
| Immunomodulation | + | Not reported | Not reported |

While the present invention is disclosed by reference to the preferred embodiments and examples detailed above, it is to be understood that these examples are intended in an illustrative rather than in a limiting sense. It is contemplated that modifications and combinations will readily occur to those skilled in the art, which modifications and combinations will be within the spirit of the invention and the scope of the following claims and its equivalent systems and methods.

What is claimed is:

1. A method for preparing progenitor cells, comprising:
obtaining a tissue sample containing myometrium from uterus, treating the tissue sample with an enzyme to remove fibrous tissue, and culturing the treated tissue sample to obtain the progenitor cells, and wherein the progenitor cells are multipotent and immunomodulatory and have negative expression of cell surface marker CD34.

2. The method of claim 1, wherein the uterus is from hysterectomies.

3. The method of claim 1, wherein the tissue sample is a biopsy sample, or an exfoliation sample.

4. The method of claim 1, which further comprises removing endometrium and serosal from the tissue sample prior to treating the tissue sample with collagenase.

5. The method of claim 1, wherein the enzyme is collagenase.

6. The method of claim 1, wherein the treated tissue sample is cultured in a serum-supplemented medium.

7. The method of claim 1, wherein the progenitor cells have positive expression of cell surface marker CD44, CD73, CD90, CD105, or any combination thereof.

8. The method of claim 1, wherein the progenitor cells have negative expression of cell surface marker CD31, CD14, CD45, CD19, HLA-DR, SidePop or any combination thereof.

9. Progenitor cells prepared according to claim 1, which have positive expression of cell surface markers CD44, CD73, CD90 and CD105, and negative expression of cell surface markers CD31, CD34, CD14, CD45, CD19, HLA-DR and SidePop.

8. The progenitor cells of claim 9, which are capable of osteogenesis, adipogenesis, chondrogenesis, and neurogenesis.

10. The progenitor cells of claim 9, which have suppressive effects on CD4 and CD8 T lymphocytes.

11. A method for treating a degenerative disease, an ischemic disease or a disease caused by abnormal immune response comprising administering progenitor cells prepared according to claim 1 to a patient subjecting the disease.

12. The method of claim 11, wherein the degenerative disease comprises Parkinson's disease, Alzheimer's disease, Huntington's disease, cerebral atrophy, cerebellar atrophy, schizophrenia and dementia.

13. The method of claim 11, wherein the ischemic disease comprises stroke, cerebral apoplexy, cerebral hemorrhage, cerebral infarction, head trauma, vascular dementia and myocardial infarction.

14. The method of claim 11, wherein the disease caused by abnormal immune response comprising autoimmune disease or graft rejection of organ transplantation.

15. The method of claim 14, wherein the autoimmune disease comprises system lupus erythematosus, multiple sclerosis, rheumatoid arthritis, type 1 diabetes mellitus, coeliac disease, Sjögren's syndrome, Hashimoto's thyroiditis, Graves' disease, and idiopathic thrombocytopenic purpura.

16. The method of claim 11, wherein the progenitor cells have positive expression of CD44, CD73, CD90 and CD105, and negative expression of CD31, CD34, CD14, CD45, CD19, HLA-DR and SidePop.
